(19) United States
(12) Patent Application Publication
(10) Pub. No.: US 2026/0272455 A1
(43) Pub. Date: Sep. 17, 2026

(54) STAPLE CARTRIDGE WITH MOVABLE KNIFE

(71) Applicant: Covidien LP, Mansfield, MA (US)

(72) Inventor: Kenneth H. WHITFIELD, North Haven, CT (US)

(21) Appl. No.: 19/162,434

(22) PCT Filed: Mar. 14, 2024
(86) PCT No.: PCT/IB2024/052455

§ 371 Date: Sep. 5, 2025

Related U.S. Application Data
(60) Provisional application No. 63/453,558, filed on Mar. 21, 2023.

(51) Int. Cl. A61B 17/072; A61B 90/00

(52) U.S. Cl. CPC A61B 17/07207; A61B 90/03; A61B 2017/07271; A61B 2017/07278; A61B 2017/07285; A61B 2090/034

(57) ABSTRACT

A surgical stapling device includes cartridge assembly having a replaceable staple cartridge. The replaceable staple cartridge includes an actuation sled assembly including an actuation sled and a knife. The knife is supported on the actuation sled for linear movement between lowered and raised positions.

This Application claims priority from U.S. Provisional Patent Application 63/453,558, 21 Mar. 2023 filed, the entire content of which is incorporated herein by reference.

### FIELD

This technology is generally related to surgical stapling devices, and more particularly, to surgical stapling devices including knife assemblies.

### BACKGROUND

Surgical stapling devices configured for endoscopic use are commonly used during surgical procedures to minimize patient trauma and reduce patient recovery times. Typically, endoscopic surgical stapling devices include a tool assembly and a drive assembly that is movable in relation to the tool assembly to actuate the tool assembly. The drive assembly includes a knife bar having a cutting blade for cutting tissue. The tool assembly includes anvil and cartridge assemblies that are coupled to each other by a pivot member and movable in relation to each other between open and clamped positions in response to movement of the drive assembly from a retracted position to an advanced position. The cartridge assembly includes a staple cartridge that includes a cartridge body that supports staples and an actuation sled that is movable through the cartridge body in response to movement of the drive assembly from the retracted and advanced position to eject the staples from the cartridge body.

Some surgical stapling devices include a staple cartridge that can be replaced after each firing of the surgical stapling device to facilitate reuse of the surgical stapling device. Other surgical stapling devices include a reload assembly that includes a staple cartridge and a drive assembly that can be replaced after each firing of the surgical stapling device to facilitate reuse of the surgical stapling device. The use of a reload assembly provides a new cutting blade for each firing of the surgical stapling device to maintain the quality of cutting. The use of replaceable staple cartridges reduces the costs associated with a surgical procedure but generally requires reuse of the cutting blade. Repeated use of the same cutting blade may cause the cutting blade to become dull reducing the quality of cutting.

A continuing need exists for a surgical stapling device that can inexpensively provide a sharpened cutting blade through multiple firings of the surgical stapling device.

### SUMMARY

Aspects of this disclosure are directed to a surgical stapling device that includes a drive assembly, and a tool assembly having a cartridge assembly with a staple cartridge. The staple cartridge includes an actuation sled assembly having an actuation sled and a knife supported on the actuation sled for linear movement between lowered and raised positions. The drive assembly and the knife are configured to move the knife between the raised and lowered position upon movement of the drive assembly from a retracted position towards an advanced position.

One aspect of this disclosure is directed to a tool assembly for a surgical stapling device that includes a drive assembly, an anvil, and a cartridge assembly. The drive assembly has a working member with an I-shape that includes a first beam, a second beam, and a vertical strut defining a cam slot connecting the first beam with the second beam. The drive assembly is movable between a drive retracted position and a drive advanced position. The cartridge assembly includes a channel that defines a cavity, a compliant member, and a staple cartridge that is releasably received within the cavity of the channel. The compliant member is positioned between the staple cartridge and the channel to facilitate movement of the staple cartridge within the cavity between a cartridge raised position and a cartridge lowered position. The staple cartridge includes a cartridge body, staples, and an actuation sled assembly. The cartridge body defines a knife slot and staple receiving pockets positioned on opposite sides of the knife slot. The staples are received within the staple receiving pockets of the cartridge body. The actuation sled assembly is movable within the cartridge body from a sled retracted position to a sled advanced position and includes an actuation sled and a knife. The actuation sled has a base portion, a central body portion that extends upwardly from the base portion, and cam members that are positioned on opposite sides of the central body portion. The central body portion of the actuation sled defines a linear guide channel defining an axis that is transverse to a longitudinal axis of the tool assembly. The knife is supported on the central body portion of the actuation sled and is movable between a lowered position and a raised position. The knife includes a first portion received within the linear guide channel of the actuation sled, a cutting blade, and a cam member. The cam member is received within the cam slot of the vertical strut of the working member of the drive assembly when the drive assembly is moved from the drive retracted position towards the drive advanced position to move the knife from the lowered position to the raised position.

In aspects of the disclosure, the knife supports a detent, and the actuation sled includes an abutment that is engaged with the detent when the knife is in the lowered position to retain the knife in the lowered position.

In some aspects of the disclosure, the actuation sled includes a resilient arm, and the abutment is supported on the resilient arm to facilitate movement of the abutment from a first position to a second position to allow the knife to move between the lowered position and the raised position.

In certain aspects of the disclosure, the central body portion of the actuation sled is movable through the knife slot of the cartridge body.

In aspects of the disclosure, the staple cartridge further includes a staple guard that is secured to a bottom of the cartridge body and has a bottom wall engaged with the base portion of the actuation sled.

In some aspects of the disclosure, the bottom wall of the staple guard includes a resilient tab, and the base portion of the actuation sled defines a recess, and the resilient tab of the staple guard is received within the recess of the actuation sled when the actuation sled assembly is in the sled advanced position to retain the actuation sled assembly in the sled advanced position.

In certain aspects of the disclosure, the cam slot is configured to move the knife from the raised position to the lowered position when the drive assembly is moved from the drive advanced position back to the drive retracted position.

In aspects of the disclosure, the base portion of the actuation sled defines a proximal cutout that is configured to receive the working member of the drive assembly when the drive assembly is moved from the drive retracted position towards the drive advanced position.

In some aspects of the disclosure, the cam member of the knife is positioned over the cutout when the actuation sled assembly is in the sled retracted position.

In certain aspects of the disclosure, the vertical strut of the working member includes a distal surface that defines a recess, and the cutting blade is at least partially received within the recess during movement of the drive assembly from the drive retracted position towards the drive advanced position.

In aspects of the disclosure, the knife includes a central portion that defines a cavity, and the actuation sled includes spaced vertical walls that are received within the cavity of the central portion of the knife to confine the knife to linear movement as the knife moves between the raised and lowered positions.

In some aspects of the disclosure, the vertical strut includes a first cam surface positioned on a first side of the cam slot and a second cam surface positioned on a second side of the cam slot, and the first cam surface and the second cam surface are configured to direct the cam member of the knife into the cam slot.

In certain aspects of the disclosure, the first cam surface is positioned to engage the cam member when the staple cartridge is positioned in the cartridge lowered position within the cavity of the channel and the second cam surface is positioned to engage the cam member when the staple cartridge is positioned in the cartridge raised position within the cavity of the channel.

Other aspects of the disclosure are directed to a surgical stapling device that includes a handle assembly, an elongate body, a drive assembly, and a tool assembly. The elongate body has a proximal portion coupled to the handle assembly and a distal portion. The drive assembly is at least partially received within the elongate body and includes a working member. The working member has an I-shape and includes a first beam, a second beam, and a vertical strut defining a cam slot connecting the first beam with the second beam. The drive assembly is movable between a drive retracted position and a drive advanced position. The tool assembly is supported on the distal portion of the elongate body and defines a longitudinal axis. The tool assembly includes an anvil and a cartridge assembly. The cartridge assembly includes a channel defining a cavity and a staple cartridge that is releasably received within the cavity of the channel. The staple cartridge includes a cartridge body, staples, and an actuation sled assembly. The cartridge body defines a knife slot and staple receiving pockets positioned on opposite sides of the knife slot. The staples are received within the staple receiving pockets of the cartridge body. The actuation sled assembly is movable within the cartridge body from a sled retracted position to a sled advanced position to eject staples from the cartridge body of the staple cartridge. The actuation sled assembly includes an actuation sled and a knife. The actuation sled has a base portion, a central body portion that extends upwardly from the base portion, and cam members that are positioned on opposite sides of the central body portion. The central body portion of the actuation sled defines a linear guide channel that defines an axis that is transverse to the longitudinal axis of the tool assembly. The knife is supported on the central body portion of the actuation sled and is movable between a lowered position and a raised position. The knife includes a first portion that is received within the linear guide channel of the actuation sled, a cutting blade, and a cam member. The cam member is received within the cam slot of the vertical strut of the working member of the drive assembly when the drive assembly is moved from the drive retracted position towards the drive advanced position to move the knife from the lowered position to the raised position.

Still other aspects of the disclosure are directed to a staple cartridge including a cartridge body, staples, and an actuation sled assembly. The cartridge body defines a knife slot and staple receiving pockets positioned on opposite sides of the knife slot.

The staples are received within the staple receiving pockets of the cartridge body, and the actuation sled assembly is movable within the cartridge body from a sled retracted position to a sled advanced position to eject staples from the cartridge body of the staple cartridge. The actuation sled assembly includes an actuation sled and a knife. The actuation sled has a base portion, a central body portion extending upwardly from the base portion, and cam members positioned on opposite sides of the central body portion. The central body portion of the actuation sled defines a linear guide channel having an axis that is transverse to the longitudinal axis of the tool assembly. The knife is supported on the central body portion of the actuation sled and is movable between a lowered position and a raised position. The knife includes a first portion received within the linear guide channel of the actuation sled, a cutting blade, and a cam member that is positioned on a proximal portion of the knife.

Other features of the disclosure will be appreciated from the following description.

### BRIEF DESCRIPTION OF THE DRAWINGS

Various aspects of the disclosed surgical stapling device are described herein below with reference to the drawings, wherein:

FIG. 1 is a side perspective view of a surgical stapling device according to aspects of the disclosure with a tool assembly of the surgical stapling device in an open position, pre-fired position;

FIG. 2 is side perspective view of the tool assembly of the surgical stapling device shown in FIG. 1 with a staple cartridge of a cartridge assembly separated from a channel of the cartridge assembly;

FIG. 3 is an exploded view of the staple cartridge of the tool assembly shown in FIG. 2;

FIG. 4 is a side perspective view of an actuation sled assembly of the staple cartridge shown in FIG. 3;

FIG. 5 is a perspective view from the proximal end of the actuation sled assembly shown in FIG. 4 with the knife in a lowered position;

FIG. 6 is an exploded view of the actuation sled assembly shown in FIG. 5;

FIG. 7 is a cross-sectional view taken along section line 7-7 of FIG. 6;

FIG. 8 is a side perspective view of a knife of the actuation sled assembly shown in FIG. 6;

FIG. 9 is a cross-sectional view taken along section line 9-9 of FIG. 5;

FIG. 10 is a perspective view from the proximal end of the staple cartridge shown in FIG. 2;

FIG. 11 is a side perspective view of a drive assembly of the surgical stapling device shown in FIG. 1;

FIG. 12 is a cross-sectional view taken through a proximal portion of the tool assembly and a distal portion of an elongate body of the surgical stapling device shown in FIG. 1 with the tool assembly of the surgical stapling device in an unclamped, pre-fired position;

FIG. 13 is a cross-sectional view taken through the proximal portion of the tool assembly and the distal portion of the elongate body of the surgical stapling device shown in FIG. 1 with the tool assembly of the surgical stapling device in a clamped, pre-fired position;

FIG. 14 is a perspective view from the proximal end of the actuation sled assembly shown in FIG. 5 with the knife in a raised position;

FIG. 15 is a cross-sectional view taken along section line 15-15 of FIG. 14;

FIG. 16 is a cross-sectional view taken through the proximal portion of the tool assembly and the distal portion of the elongate body of the surgical stapling device shown in FIG. 1 with the tool assembly of the surgical stapling device in a clamped, pre-fired position;

FIG. 17 is a cross-sectional view taken through the proximal portion of the tool assembly and the distal portion of the elongate body of the surgical stapling device shown in FIG. 1 with the tool assembly of the surgical stapling device in a clamped, fired position and the drive assembly in an advanced position;

FIG. 18 is a cross-sectional view taken through the distal portion of the tool assembly of the surgical stapling device shown in FIG. 1 with the tool assembly of the surgical stapling device in a clamped, fired position as the drive assembly begins to return from advanced position towards the retracted position;

FIG. 19 is a cross-sectional view taken through the proximal portion of the tool assembly and the distal portion of the elongate body of the surgical stapling device shown in FIG. 1 with the tool assembly of the surgical stapling device in a clamped, fired position and the drive assembly in a retracted position;

FIG. 20 is an exploded view of an alternate version of the tool assembly of the surgical stapling device shown in FIG. 1;

FIG. 21 is an exploded, side perspective view of an alternate version of the drive assembly of the stapling device shown in FIG. 1 and the actuation sled assembly of the tool assembly shown in FIG. 20;

FIG. 22 is a cross-sectional view taken through the proximal portion of the tool assembly shown in FIG. 20 and the distal portion of the drive assembly shown in FIG. 21 with the tool assembly in the clamped position and the staple cartridge in the raised position within the channel of the cartridge assembly of the tool assembly;

FIG. 23 is a cross-sectional view taken along section line 23-23 of FIG. 22;

FIG. 24 a cross-sectional view taken through the proximal portion of the tool assembly shown in FIG. 20 and the distal portion of the drive assembly shown in FIG. 21 with the tool assembly in the clamped position and the staple cartridge in the lowered position within the channel of the cartridge assembly of the tool assembly; and

FIG. 25 is a cross-sectional view taken along section line 25-25 of FIG. 22.

### DETAILED DESCRIPTION

The disclosed surgical stapling device will now be described in detail with reference to the drawings in which like reference numerals designate identical or corresponding elements in each of the several views. However, it is to be understood that disclosed aspects of the surgical stapling device are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

In this description, the term “proximal” is used generally to refer to that portion of the device that is closer to a clinician during use of the device in its customary fashion, while the term “distal” is used generally to refer to that portion of the device that is farther from the clinician during use of the device in its customary fashion. In addition, directional terms such as front, rear, upper, lower, top, bottom, and similar terms are used to assist in understanding the description and are not intended to limit the disclosure. Further, the term “clinician” is used generally to refer to medical personnel including doctors, nurses, surgeons, and support personnel.

The disclosure is directed to a surgical stapling device that includes a drive assembly, and a tool assembly having a cartridge assembly with a staple cartridge. The staple cartridge has an actuation sled assembly that includes an actuation sled and a knife that is supported on the actuation sled for linear movement between lowered and raised positions. The drive assembly and the knife are configured to move the knife between the raised and lowered position upon movement of the drive assembly from a retracted position towards an advanced position.

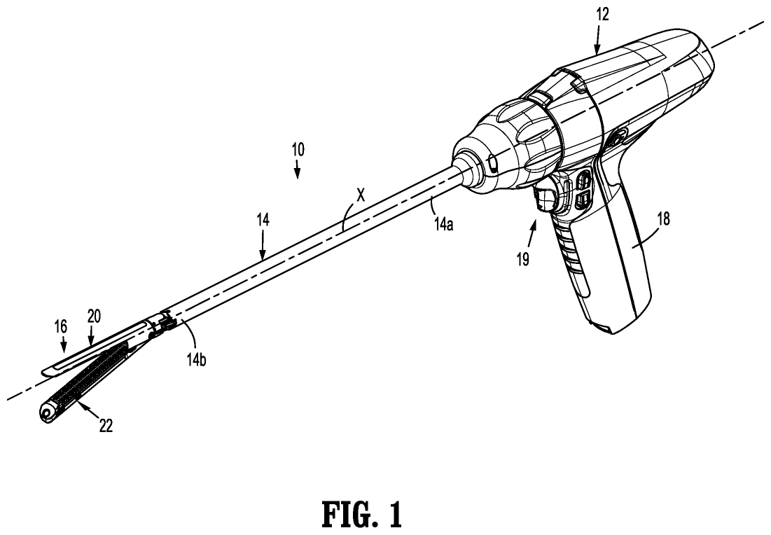

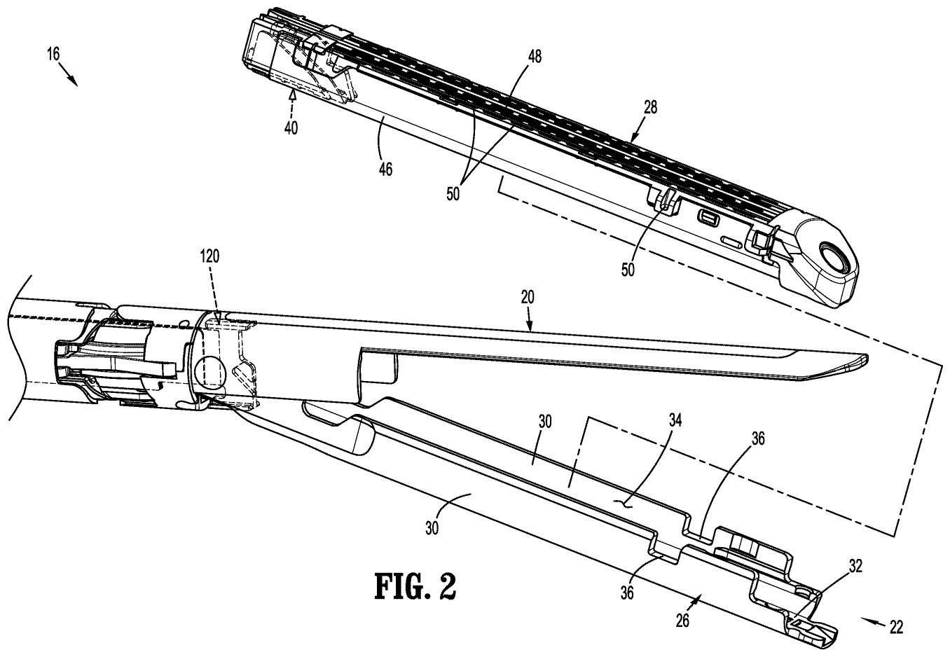

FIGS. 1 and 2 illustrate a surgical stapling device shown generally as stapling device 10 that includes a handle assembly 12, an adapter assembly 14, and a tool assembly 16. The handle assembly 12 is powered and includes a stationary handgrip 18 and an actuation button or buttons 19. The actuation buttons 19 are operable to actuate various functions of the tool assembly 16 via the adapter assembly 14, i.e., approximation of the tool assembly 16, and stapling and cutting of tissue. In certain aspects of the disclosure, the handle assembly 12 supports batteries (not shown) that provide power to the handle assembly 12 to operate the stapling device 10. Although the stapling device 10 is illustrated as a powered stapling device, it is envisioned that the advantages of this disclosure are suitable for use with manually powered surgical stapling devices as well as robotically controlled stapling devices. U.S. Pat. No. 5,865,361 describes a stapling device that includes exemplary aspects of a manually powered stapling device.

The adapter assembly 14 defines a longitudinal axis “X” and includes a proximal portion 14a and a distal portion 14b. The proximal portion 14a of the adapter assembly 14 is coupled to the handle assembly 12, and the distal portion 14b of the adapter assembly 14 supports the tool assembly 16. In aspects of the disclosure, the tool assembly 16 is pivotable coupled to the distal portion 14b of the adapter assembly to facilitate articulation of the tool assembly 16 in relation to the adapter assembly 14.

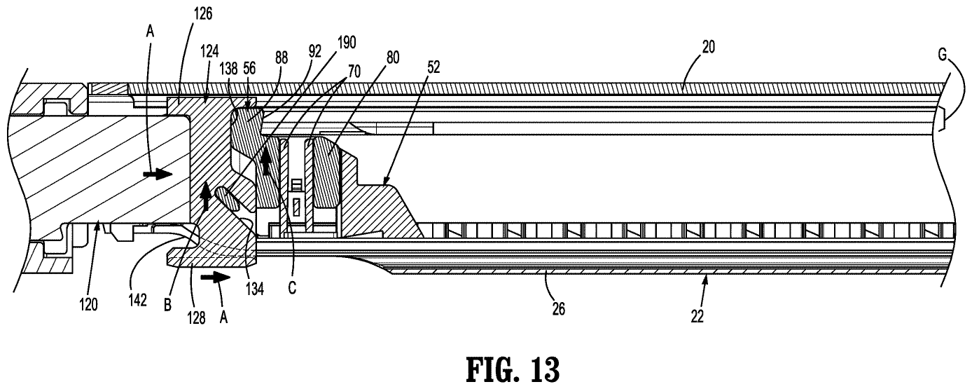

The tool assembly 16 includes an anvil 20 and a cartridge assembly 22. In aspects of the disclosure, the cartridge assembly 22 is pivotably supported in relation to the anvil 20 to facilitate movement of the tool assembly 16 between an unclamped position (FIG. 1) and a clamped position (FIG. 13).

FIG. 2 illustrates the cartridge assembly 22 which includes a channel member 26 and a staple cartridge 28. The channel member 26 includes side walls 30 and a bottom wall 32 that define a cavity 34. The staple cartridge 28 is removably received within the cavity 34 of the channel member 26 and is replaceable to facilitate reuse of the stapling device 10. Each of the side walls 30 of the channel member 26 has an upper end that defines a recess 36.

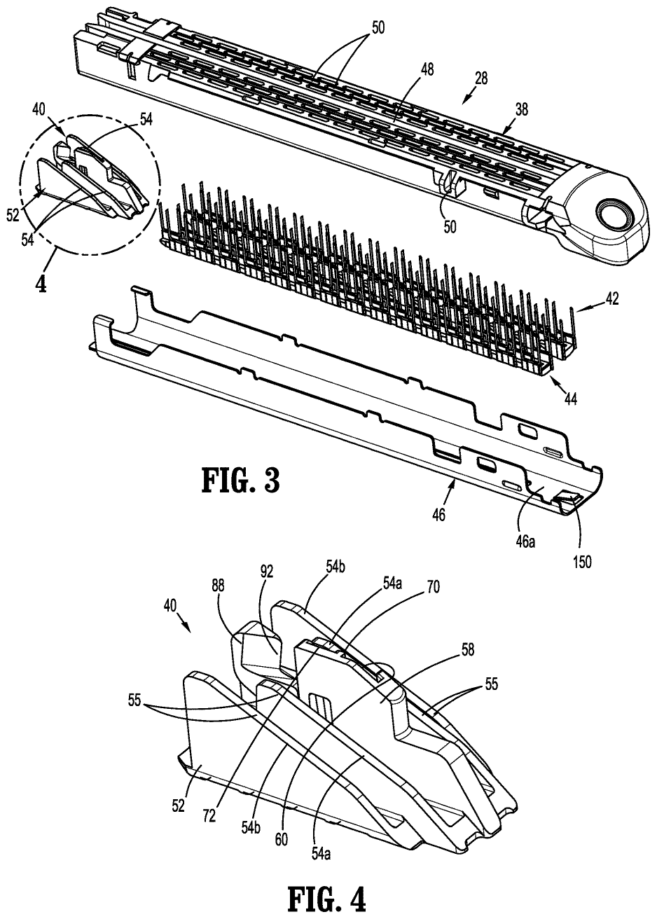

FIG. 3 illustrates the staple cartridge 28 which includes a cartridge body 38, an actuation sled assembly 40, staples 42, pushers 44, and a staple guard 46. The cartridge body 38 defines a knife slot 48 and staple receiving pockets 50 that are positioned on each side of the knife slot 48. In aspects of the disclosure, the staple receiving pockets 50 are aligned in two or more rows on opposite sides of the knife slot 48. In aspects of the disclosure, the knife slot 48 is centrally located in the cartridge body 38. However, it is envisioned that the knife slot 48 could be offset from the central axis of the cartridge body 38.

The cartridge body 38 includes laterally extending protrusions 51 (only one is shown) that are received within the recesses 36 (FIG. 2) of the channel member 26 to properly position the staple cartridge 28 within the cavity 34 of the channel member 26. The staples 42 and the pushers 44 are received within the staple receiving pockets 50 of the cartridge body 38, and the staple guard 46 is secured to the bottom of the cartridge body 38 to retain the staples 42 and pushers 44 within the cartridge body 38. The actuation sled assembly 40 is movable through the cartridge body 38 from a retracted position to an advanced position and includes an actuation sled 52 having angled inner and outer cam members 54a, 54b. The cam members 54a, 54b have angled cam surfaces 55 that are positioned to sequentially engage the pushers 44 as the actuation sled 52 moves through the cartridge body 38 to lift the pushers 44 within the staple receiving pockets 50 and eject the staples 42 from the cartridge body 38.

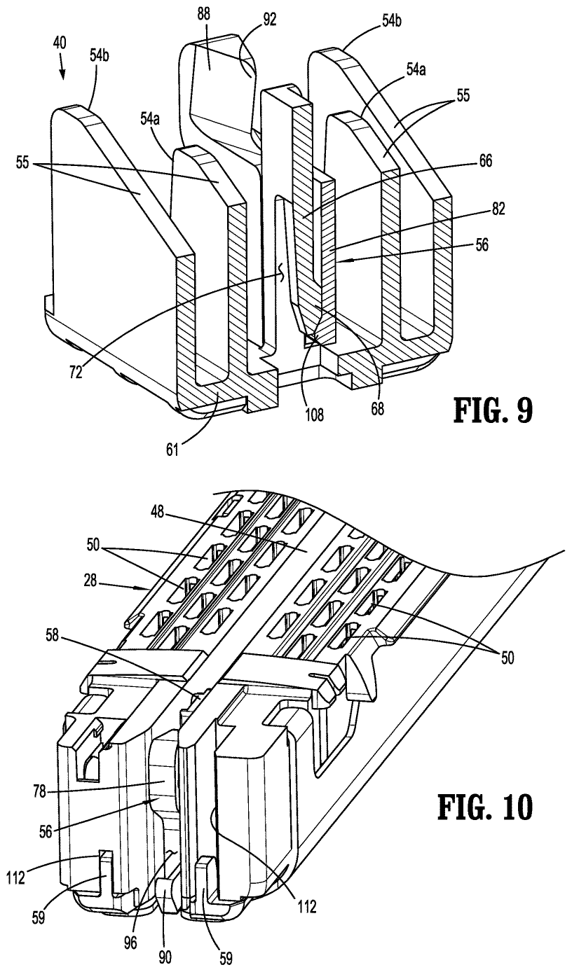

FIGS. 4-9 illustrate the actuation sled assembly 40 which includes the actuation sled 52 and a knife 56. The actuation sled 52 includes the cam members 54a, 54b, a central body portion 58, and fins 59. The cam members 54a, 54b are positioned on opposite sides of the central body portion 58, and each of the fins 59 is longitudinally aligned and positioned proximally of one of the inner cam members 54a.

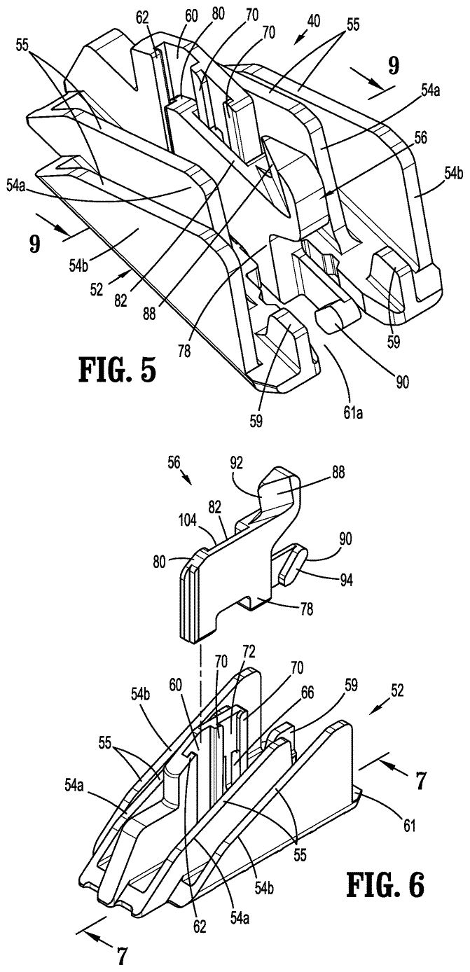

The actuation sled 52 includes a base portion 61 that is supported on a bottom wall 46a of the staple guard 46 and defines a cutout 61a (FIG. 5). The central body portion 58 and the cam members 54a, 54b extend upwardly from the base portion 61 of the actuation sled 52 with the central body portion 58 positioned between the inner cam members 54a. The central body portion 58 is received within the knife slot 48 of the cartridge body 38 as the actuation sled assembly 40 moves within the cartridge body 38 from the retracted position towards the advanced position. The central body portion 58 defines a guide channel 60 (FIG. 6) and includes a lip 62 that extends partially over the guide channel 60. The central body portion 58 also includes a resilient arm 66 that supports an abutment 68. In aspects of the disclosure, the resilient arm 66 is integrally formed with the central body portion 58 and is supported on the central body portion 58 in cantilevered fashion to facilitate inward and outward movement of the abutment 68. In aspects of the disclosure, the central body portion 58 includes spaced vertical walls 70 that are positioned proximally of the guide channel 60 and are spaced from each other to define a recess 72. The resilient arm 66 is received within the recess 72 and is movable between a first position extending from the recess 72 and a second position positioned within the recess 72.

The knife 56 includes a body 76 having a proximal portion 78, a distal portion 80, and a central portion 82. The distal portion 80 of the knife 56 is received within the guide channel 60 of the actuation sled 52 and includes a distal flange 84 that is engaged by the lip 62 of the of the central body portion 58 of the actuation sled 52 to retain the distal portion 80 of the knife 56 within the guide channel 60. The guide channel 60 of the actuation sled 52 and the distal portion 80 of the knife 56 are configured and dimensioned to facilitate vertical movement of the knife 56 in relation to the actuation sled 52 between lowered and raised positions as described in further detail below.

The proximal portion 78 of the knife 56 includes a cutting blade 88 and a cam member 90. The cutting blade 88 forms the upper section of the proximal portion 78 of the knife 56 and includes a distally facing cutting edge 92. The cam member 90 is positioned on a lower section of the proximal portion 78 of the knife 56 and includes an elongate, angled protrusion 94. It is envisioned that the protrusion 94 of the cam member 90 can have a variety of configurations including a circular configuration. In aspects of the disclosure, the proximal portion 78 is supported on the central portion 82 of the knife 56 in cantilevered fashion. The proximal portion 78 of the knife 56 defines a cavity 96 that is positioned between the cutting blade 88 and the cam member 90.

The central portion 82 of the knife 56 extends between a proximal wall 100 (FIG. 8) of the distal portion 80 of the knife 56 and a distal wall 102 of the proximal portion 78 of the knife 56. The proximal wall 100, the distal wall 102, and the central portion 82 define a cavity 104 that receives the spaced vertical walls 70 of the actuation sled 52 when the distal portion 80 of the knife 56 is received in the guide channel 60 of the actuation sled 52. When the spaced vertical walls 70 of the actuation sled 52 are received within the cavity 104 of the knife 56, the distal wall 102 of the proximal portion 78 of the knife 56 is in sliding engagement with a distal surface of one of the vertical walls 70 of the actuation sled 52 and the proximal wall 100 (FIG. 8) of the distal portion 80 of the knife 56 is in sliding engagement with a proximal surface of the other of the vertical walls 70 of the actuation sled 52. The central portion 82 of the knife 56 includes a detent 108 that projects into the cavity 104 of the knife 56.

FIG. 10 illustrates the proximal portion of the staple cartridge 28 with the actuation sled assembly 40 in the retracted position within the cartridge body 38. As illustrated, the cam member 90 of the knife 56 is positioned in a proximal end of the knife slot 48 of the cartridge body 38. In this position, the abutment 68 on the resilient arm 66 of the actuation sled 52 is engaged with the detent 108 on the central portion 82 of the knife 56 to retain the knife 56 in the lowered position, the central body portion 58 of the actuation sled 52 is received in the knife slot 48 of the cartridge body 38, and the fins 59 are received in channels 112 of the cartridge body 38.

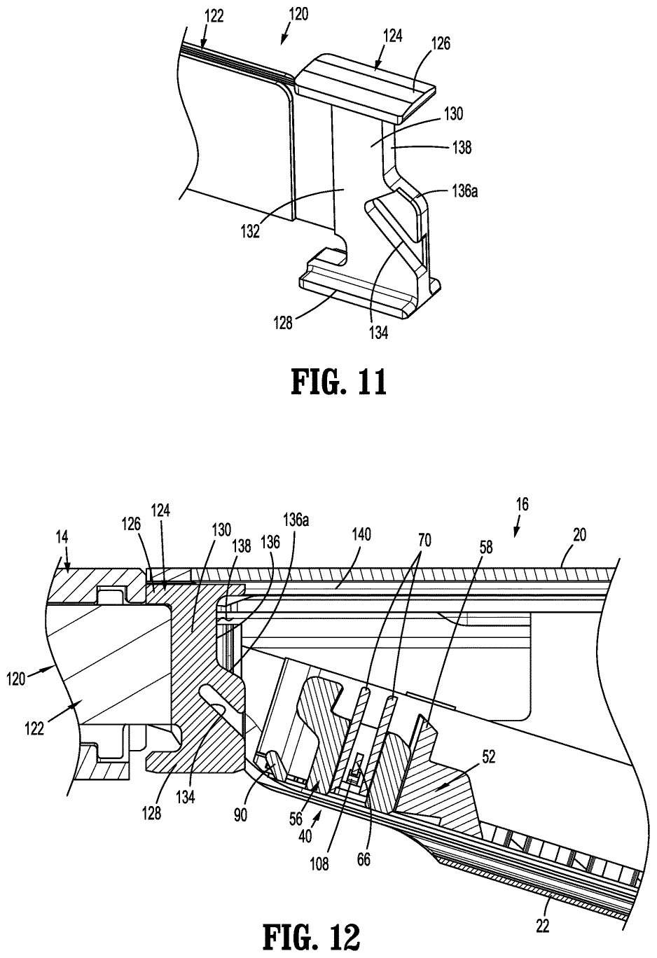

FIG. 11 illustrates a distal portion of a drive assembly 120 of the stapling device 10 (FIG. 1) which includes a flexible drive beam 122 and a working member 124. The flexible drive beam 122 includes stacked sheets of material that have distal ends secured to the working member 124. The working member 124 has an I-beam configuration and includes a first beam 126, a second beam 128, and a vertical strut 130 that secures the first and second beams 126, 128 together at a fixed distance from each other. The vertical strut 130 has a side wall 132 that defines a cam slot 134 and a distal surface 136 that includes an angled portion 136a that is angled upwardly and proximally at a position between the cam slot 134 and the first beam 126 to define a recess 138 between the first beam 126 and the angled portion 136a. The drive assembly 120 is movable through the cartridge body 38 from a retracted position to an advanced position to advance the actuation sled assembly 40 through the cartridge body 38 from its retracted position to its advanced position. As the drive assembly 120 moves through the cartridge body 38, the vertical strut 130 of the working member 124 moves through the knife slot 48 of the cartridge body 38 and the first and second beams 126, 128 engage the anvil 20 and the cartridge assembly 22, respectively, to move the tool assembly 16 from the unclamped position to the clamped position and maintain a maximum tissue gap between the anvil 20 and the cartridge assembly 22.

FIG. 12 illustrates the proximal portion of the tool assembly 16 in the unclamped position. When the tool assembly 16 is in the unclamped position, the cartridge assembly 22 is pivoted away from the anvil 20 and the actuation sled assembly 40 is in its retracted position in the proximal portion of the cartridge body 38 of the cartridge assembly 22. The drive assembly 120 is also positioned in its retracted position spaced proximally of the cartridge assembly 22, and the first beam 126 of the drive assembly 120 is partially received within a channel 140 defined within the anvil 20. The cam member 90 of the knife 56 is positioned at the proximal end of the cartridge assembly 22 adjacent to the cam slot 134 of the working member 124 of the drive assembly 120, and the knife 56 is in the lowered position.

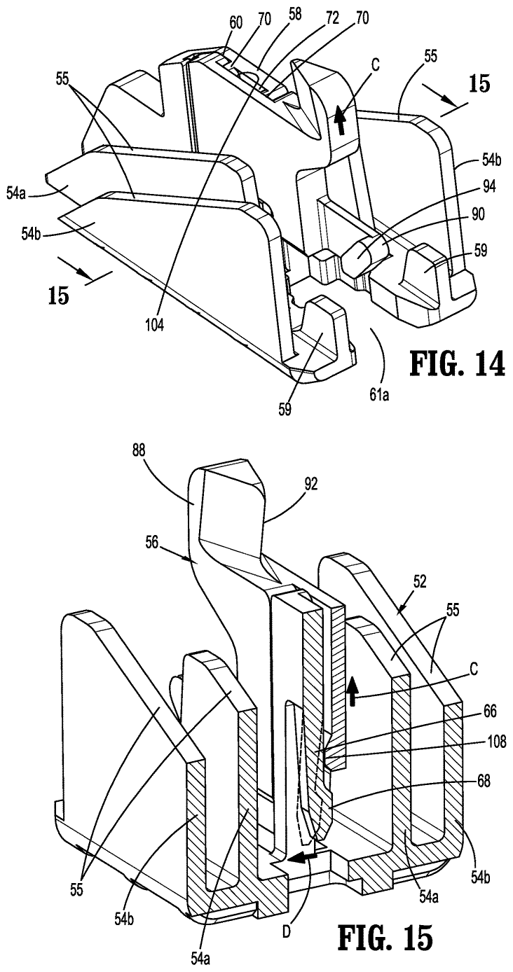

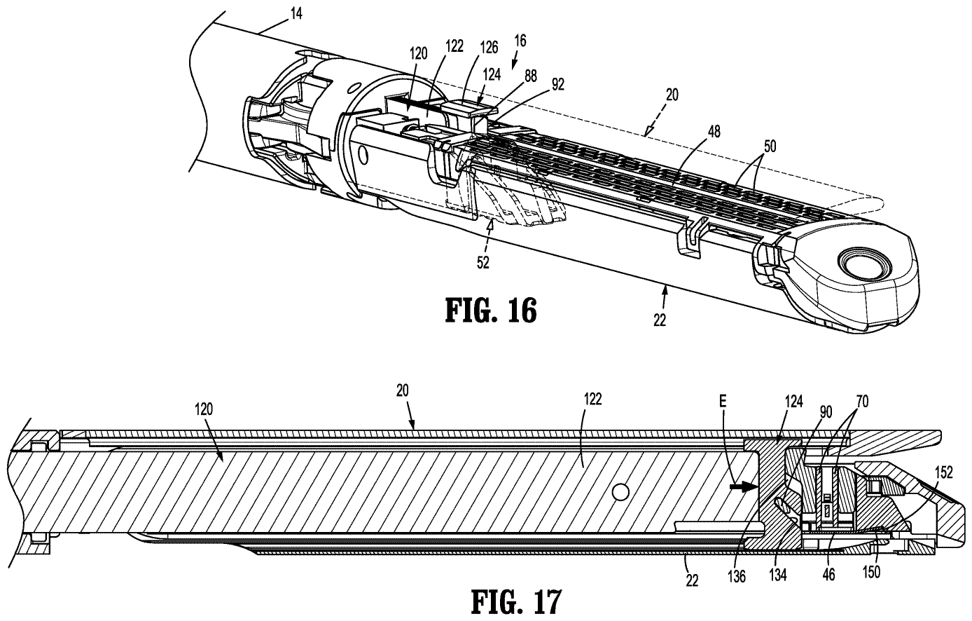

FIGS. 13-16 illustrate the tool assembly 16 of the stapling device 10 (FIG. 1) as the tool assembly 16 is moved to the clamped position. When the drive assembly 120 is moved in the direction indicated by arrows “A” in FIG. 13 from its retracted position towards its advanced position, the second beam 128 of the working member 124 of the drive assembly 120 engages a bend 142 (FIG. 13) in the channel 26 of the cartridge assembly 22 and pivots the cartridge assembly 22 in relation to the anvil 20 to the clamped position, and the working member 124 moves into the cutout 61a (FIG. 14) defined in the base portion 61 of the actuation sled 52. As the cartridge assembly pivots in relation to the anvil 20 towards the clamped position, the cam member 190 of the knife 56 is received in the cam slot 134 of the working member 124 of the drive assembly 120 and is cammed upwardly in the cam slot 134 in the direction indicated by arrow “B” to lift the knife 56 from the lowered position to the raised position in the direction indicated by arrow “C”. In the raised position of the knife 56, the cutting edge 92 of the knife 56 extends from the knife slot 48 of the cartridge body 38 across a tissue gap “G” defined between the anvil 20 and the cartridge assembly 22, and the proximal portion of the cutting blade 88 of the knife 52 is received within the recess 138 of the working member 124 of the drive assembly 120. As the knife 56 moves from the lowered position to the raised position, the detent 108 on the central portion 82 of the knife 56 engages the abutment 68 on the resilient arm 66 of the actuation sled 52 and forces the abutment 68 in the direction indicated by arrow “D” in FIG. 15 to allow the knife 56 to move upwardly past the abutment 68 to the raised position.

FIG. 17 illustrates drive assembly 120 of the stapling device 10 (FIG. 1) as the drive assembly 120 moves in the direction indicated by arrow “E” from its clamped position to the advanced position to fire the tool assembly 16. As the drive assembly 120 moves towards its advanced position, the distal surface 136 of the working member 124 of the drive assembly 120, which is in abutting relation with the knife 56, advances the actuation sled assembly 40 towards its advanced position to eject the staples 42 from the cartridge body 38 and cut tissue clamped between the anvil 20 and the cartridge assembly 22. In aspects of the disclosure, the bottom wall 46a of the staple guard 46 includes a resilient tab 150 (FIG. 3) that extends upwardly from the bottom wall 46a into the path of the actuation sled assembly 40. The resilient tab 150 is supported at its proximal end on the bottom wall 46a in cantilevered fashion. When the actuation sled 52 passes over the resilient tab 150, the resilient tab 150 is deformed downwardly and subsequently snaps into a recess 152 formed on the bottom of the actuation sled 52. When the tab 150 is received in the recess 152 of the actuation sled 52, the actuation sled assembly 40 is prevented from moving proximally within the cartridge body 38 of the cartridge assembly 22.

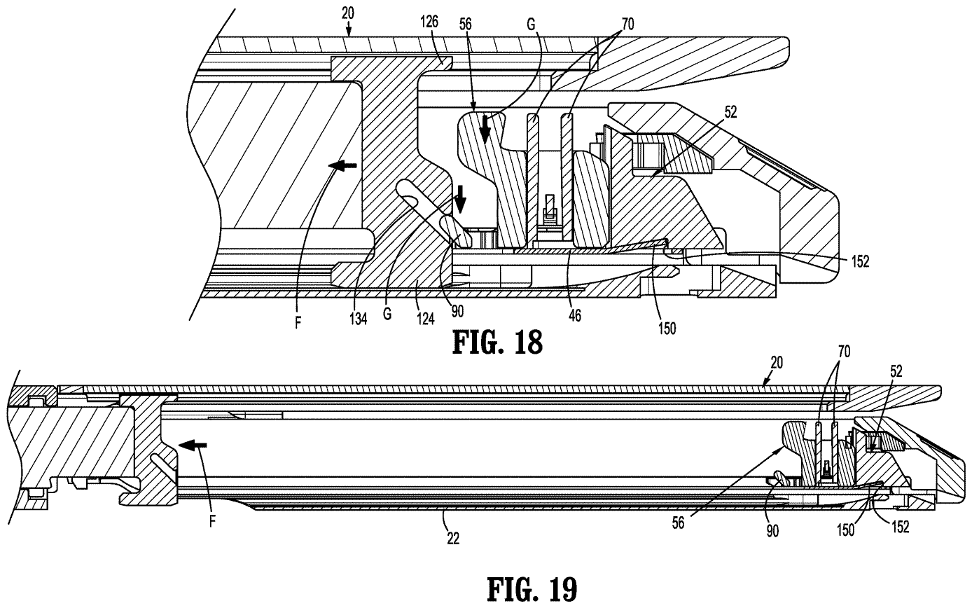

FIGS. 18 and 19 illustrate the tool assembly 16 of the stapling device 10 (FIG. 1) as the drive assembly 120 is moved in the direction indicated by arrow “F” from the advanced position to the retracted position after the stapling device 10 is fired. When the drive assembly 120 is retracted after firing, the actuation sled assembly 40 is retained in its advanced position by the tab 150 on the staple guard 46. As such, the drive assembly 120 moves independently of the actuation sled 52 from the advanced position towards the retracted position to move the cam slot 134 of the working member 124 of the drive assembly 120 in relation to the cam member 90 of the knife 56. This movement cams the knife 56 downwardly in the direction indicated by arrows “G” in FIG. 19 to move the knife 56 from the raised position back to the lowered position and disengage the working member 124 of the drive assembly 120 from the actuation sled assembly 40. As described above, the knife 56 is retained in the lowered position by the engagement of the detent 108 on the central portion 82 of the knife 56 with the abutment 68 on the resilient arm 66 of the actuation sled 52.

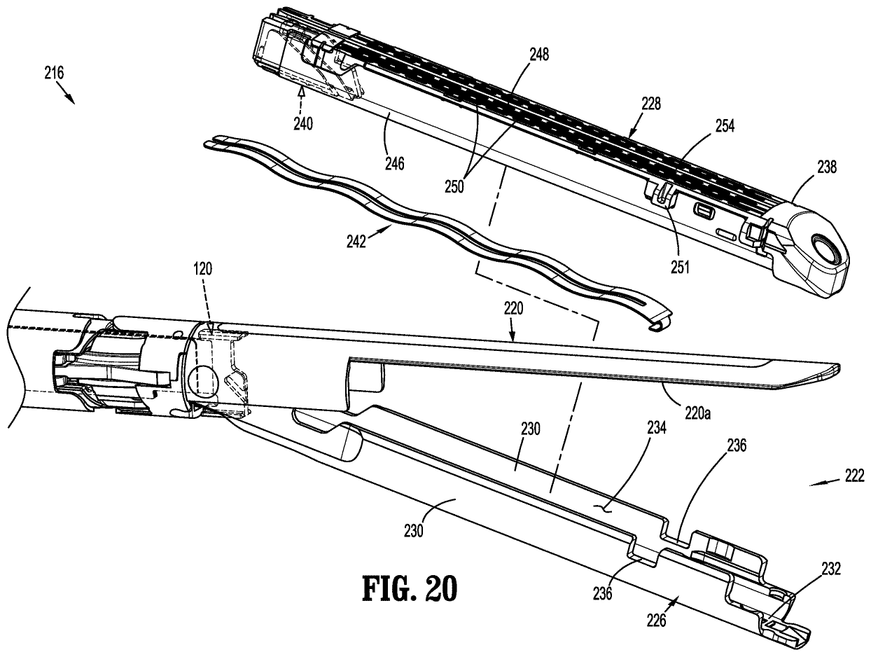

FIGS. 20-25 illustrate an alternate version of the tool assembly 16 (FIG. 2) of the stapling device 10 (FIG. 1) shown generally as tool assembly 216. The tool assembly 216 is like the tool assembly 16 (FIG. 2) and includes an anvil 220 and a cartridge assembly 222. The cartridge assembly 222 includes a channel member 226 and a staple cartridge 228. The channel member 226 includes side walls 230 and a bottom wall 232 that define a cavity 234. The staple cartridge 228 is removably received within the cavity 234 of the channel member 226 and is replaceable to facilitate reuse of the stapling device 10 (FIG. 1). Each of the side walls 230 of the channel member 226 has an upper end that defines a recess 236.

The staple cartridge 228 is identical to the staple cartridge 28 except for minor modifications to the actuation sled 240 which are described below. As described above regarding staple cartridge 28 (FIG. 3), the staple cartridge 228 includes a cartridge body 238 that defines a knife slot 248 and staple receiving pockets 250 that are positioned on each side of the knife slot 248. The cartridge body 38 includes laterally extending protrusions 251 (only one is shown) that are received within the recesses 236 (FIG. 2) of the channel member 226 to properly position the staple cartridge 228 within the cavity 234 of the channel member 226. The staple cartridge 228 also includes a staple guard 246 that is secured to a bottom of the cartridge body 238 to retain staples and pushers (not shown) within the cartridge body 238 during shipping and handling of the staple cartridge 228.

The tool assembly 216 differs from the tool assembly 16 (FIG. 2) in that the tool assembly 216 includes a compliant member 242 that can be in the form of a wave spring and is secured to the bottom wall 232 of the channel member 226 to support the staple cartridge 228 within the cavity 234 of the channel member 234. The compliant member 236 is compressible to facilitate movement of the staple cartridge 228 within the cavity 234 of the channel member 226 in relation to the anvil 220 between a cartridge raised position and a cartridge lowered position to accommodate tissues of different thicknesses. The compliant member 242 can be formed from a variety of different compliant materials and functions to allow the staple cartridge 228 to float within the cavity 234 of the channel such that a tissue gap defined between the tissue engaging surface 220a of the anvil 220 and a tissue engaging surface 254 of the staple cartridge 228 of the cartridge assembly 222 can change when the tool assembly 216 is in the clamped position and/or fired to compensate for the different tissue thicknesses. In some aspects of the disclosure, the compliant member 242 is positioned along opposite sides of a knife slot 248 defined in the bottom wall 232 of the channel member 226. Although shown as a wave spring, the compliant member 242 can be formed from compressible pads or the like.

The actuation sled assembly 240 of the staple cartridge 228 is like the actuation sled assembly 40 (FIG. 5) and includes an actuation sled 252 and a knife 256 that is supported on the actuation sled 252 and is movable along a linear path between lowered and raised positions. The actuation sled 252 differs from the actuation sled 52 (FIG. 6) in that the cam member 290 has a circular configuration. The cam member 290 is supported on the proximal end of the proximal portion 278 of the knife 256 in cantilevered fashion.

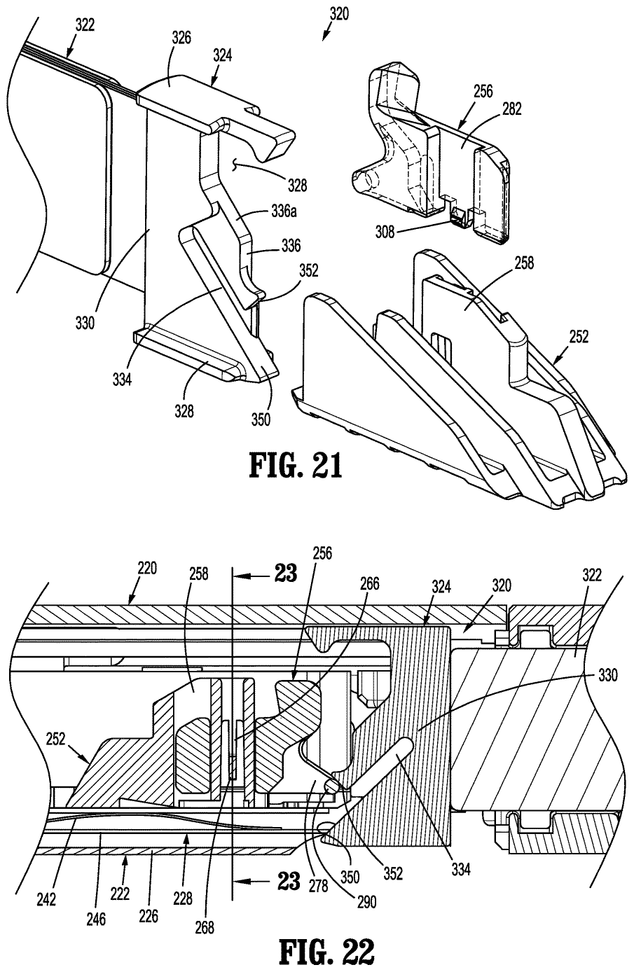

FIG. 21 illustrates the distal portion of an alternate version of the drive assembly 120 (FIG. 11) of the stapling device 10 shown generally as drive assembly 320 which includes a flexible drive beam 322 and a working member 324. The flexible drive beam 322 includes stacked sheets of material that have distal ends secured to the working member 324. The working member 324 has an I-beam configuration and includes a first beam 326, a second beam 328, and a vertical strut 330 that secures the first and second beams 326, 328 together at a fixed distance from each other. The vertical strut 330 has a side wall 332 that defines a cam slot 334 and a distal surface 336 that includes an angled portion 336a that is angled upwardly and proximally at a position between the cam slot 334 and the first beam 326 to define a recess 338 between the first beam 326 and the angled portion 336a. The drive assembly 320 is movable through the cartridge body 238 from a drive retracted position to a drive advanced position to advance the actuation sled assembly 240 through the cartridge body 238 from a sled retracted position to a sled advanced position. As the drive assembly 320 moves through the cartridge body 238, the vertical strut 330 of the working member 324 moves through the knife slot 348 of the cartridge body 338 and the first and second beams 326, 328 engage the anvil 320 and the cartridge assembly 322, respectively, to move the tool assembly 316 from the unclamped position to the clamped position. The upper and lower beams 326 and 328 engage the anvil 220 and the channel 226 of the cartridge assembly 222 to set a maximum tissue gap between the tissue engaging surfaces 220a, 254 of the anvil 220 and the staple cartridge 228 of the cartridge assembly 22.

The working member 324 of the drive assembly 320 differs from the working member 124 (FIG. 11) in that the working member 324 is configured to guide the cam member 290 of the knife 256 into the cam slot 334 when the knife 256 is in both the cartridge lowered position and the cartridge raised position, or at any position between the cartridge raised and lowered positions. More specifically, the vertical strut 330 of the working member 324 of the drive assembly 320 includes a first cam surface 350 and a second cam surface 352. The first cam surface 350 is positioned below the cam slot 334, distally of the second beam 328 and is contiguous with a surface of the vertical strut 332 defining one side of the cam slot 334. The second cam surface 352 is positioned above the cam slot 334 and is angled downwardly in the proximal direction towards the cam slot 334.

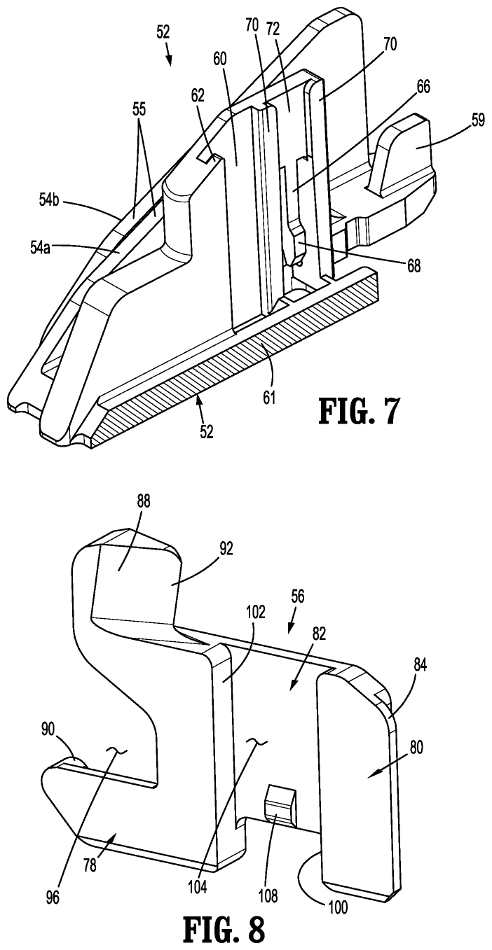

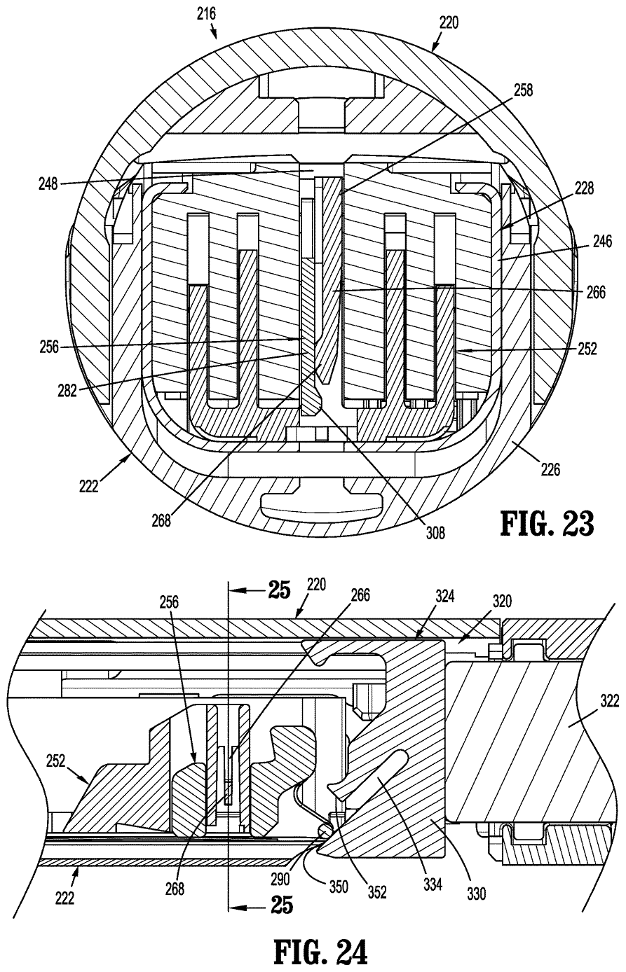

FIGS. 22 and 23 illustrate the tool assembly 216 in a clamped position with the staple cartridge 228 in the cartridge raised position within the cavity 234 (FIG. 20) of the channel 226 of the cartridge assembly 222. As described above regarding the actuation sled 52 (FIG. 7) and the knife 56 (FIG. 8), the actuation sled 252 includes a central body portion 258 having a resilient arm 266 that supports an abutment 268, and the knife 256 includes a central portion 282 having a detent 308. In aspects of the disclosure, the resilient arm 266 is integrally formed with the central body portion 258 of the actuation sled 252 and is supported on the central body portion 258 in cantilevered fashion to facilitate inward and outward movement of the abutment 268. In the clamped position, the detent 308 of the knife 256 is positioned below the abutment 268 of the actuation sled 252 to retain the knife in the lowered position. When the staple cartridge 228 is in the cartridge raised position within the channel 226 of the cartridge assembly 222 (FIGS. 22 and 23), such as when thin tissue is clamped between the staple cartridge 228 and the anvil 220, the knife 256 is in a first lowered position in relation to the actuation sled 252. In the first lowered position, the cam member 290 of the knife 256 is engaged with the second cam surface 352 of the working member 324 of the drive assembly 320 and the detent 308 of the knife 256 is positioned slightly below the abutment 268 of the actuation sled 252. When the drive assembly 320 is advanced from the drive clamped position towards the drive advanced position, the second cam surface 352 of the working member 324 of the drive assembly 320 directs the cam member 290 downwardly into the cam slot 334 of the working member 324 of the drive assembly 320.

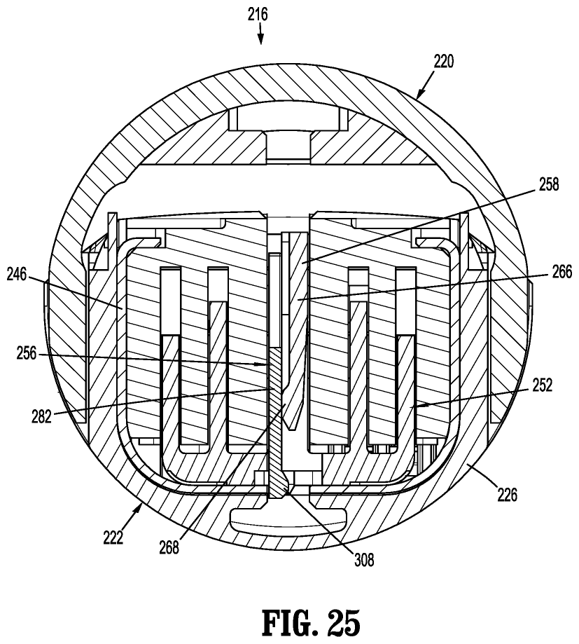

FIGS. 24 and 25 illustrate the tool assembly 216 in a clamped position with the staple cartridge 228 in the cartridge lowered position within the cavity 234 (FIG. 20) of the channel 226 of the cartridge assembly 222. When the staple cartridge 228 is in the cartridge lowered position within the channel 226 of the cartridge assembly 222 (FIGS. 22 and 23), such as when thick tissue is clamped between the staple cartridge 228 and the anvil 220, the knife 256 is in a second lowered position in relation to the actuation sled 252. In the second lowered position, the cam member 290 of the knife 256 is engaged with the first cam surface 350 of the working member 324 of the drive assembly 320 and the detent 308 of the knife 256 is positioned further below the abutment 268 of the actuation sled 252. When the drive assembly 320 is advanced from the drive clamped position towards the drive advanced position, the first cam surface 352 of the working member 324 of the drive assembly 320 directs the cam member 290 upwardly into the cam slot 334 of the working member 324 of the drive assembly 320.

The tool assembly functions in a manner like that of the tool assembly 16 (FIG. 2). As such operation of the tool assembly 216 will not be described in further detail herein.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A tool assembly for a staple stapling device, the tool assembly comprising:
a drive assembly including a working member having an I-shape and including a first beam, a second beam, and a vertical strut connecting the first beam with the second beam, the vertical strut defining a cam slot, the drive assembly movable between a drive retracted position and a drive advanced position;
an anvil; and
a cartridge assembly including a channel defining a cavity, a compliant member, and a staple cartridge that is releasably received within the cavity of the channel, the compliant member positioned between the staple cartridge and the channel to facilitate movement of the staple cartridge within the cavity between a cartridge raised position and a cartridge lowered position, the staple cartridge including:
a cartridge body defining a knife slot and staple receiving pockets, the staple receiving pockets positioned on opposite sides of the knife slot;
staples received within the staple receiving pockets of the cartridge body; and
an actuation sled assembly movable within the cartridge body from a sled retracted position to a sled advanced position, the actuation sled assembly including:
an actuation sled having a base portion, a central body portion extending upwardly from the base portion, and cam members positioned on opposite sides of the central body portion, the central body portion of the actuation sled defining a linear guide channel defining an axis that is transverse to a longitudinal axis of the tool assembly; and
a knife supported on the central body portion of the actuation sled and movable between a lowered position and a raised position, the knife including a first portion received within the linear guide channel of the actuation sled, a cutting blade, and a cam member, the cam member received within the cam slot of the vertical strut of the working member of the drive assembly when the drive assembly is moved from the drive retracted position towards the drive advanced position to move the knife from the lowered position to the raised position.

2. The tool assembly of claim 1, wherein the knife supports a detent, and the actuation sled includes an abutment that is engaged with the detent when the knife is in the lowered position to retain the knife in the lowered position.

3. The tool assembly of claim 2, wherein the actuation sled includes a resilient arm, and the abutment is supported on the resilient arm, the resilient arm facilitating movement of the abutment from a first position to a second position to allow the knife to move between the lowered position and the raised position.

4. The tool assembly of claim 1, wherein the central body portion of the actuation sled is movable through the knife slot of the cartridge body.

5. The tool assembly of claim 1, wherein the staple cartridge further includes a staple guard secured to a bottom of the cartridge body, the staple guard having a bottom wall engaged with the base portion of the actuation sled.

6. The tool assembly of claim 5, wherein the bottom wall of the staple guard includes a resilient tab, and the base portion of the actuation sled defines a recess, the resilient tab of the staple guard received within the recess of the actuation sled when the actuation sled assembly is in the sled advanced position to retain the actuation sled assembly in the sled advanced position.

7. The tool assembly of claim 6, wherein the cam slot is configured to move the knife from the raised position to the lowered position when the drive assembly is moved from the drive advanced position back to the drive retracted position.

8. The tool assembly of claim 1, wherein the base portion of the actuation sled defines a proximal cutout that is configured to receive the working member of the drive assembly when the drive assembly is moved from the drive retracted position towards the drive advanced position.

9. The tool assembly of claim 8, wherein the cam member of the knife is positioned over the cutout when the actuation sled assembly is in the sled retracted position.

10. The tool assembly of claim 1, wherein the vertical strut of the working member includes a distal surface that defines a recess, the cutting blade at least partially received within the recess during movement of the drive assembly from the drive retracted position towards the drive advanced position.

11. The tool assembly of claim 1, wherein the knife includes a central portion that defines a cavity, and the actuation sled includes spaced vertical walls that are received within the cavity of the central portion of the knife to confine the knife to linear movement as the knife moves between the raised and lowered positions.

12. The tool assembly of claim 1, wherein the vertical strut includes a first cam surface positioned on a first side of the cam slot and a second cam surface positioned on a second side of the cam slot, the first cam surface and the second cam surface configured to direct the cam member of the knife into the cam slot.

13. The tool assembly of claim 12, wherein the first cam surface is positioned to engage the cam member when the staple cartridge is positioned in the cartridge lowered position within the cavity of the channel and the second cam surface is positioned to engage the cam member when the staple cartridge is positioned in the cartridge raised position within the cavity of the channel.

14. A surgical stapling device comprising:
a handle assembly;
an elongate body having a proximal portion and a distal portion, the proximal portion coupled to the handle assembly;
a drive assembly at least partially received within the elongate body, the drive assembly including a working member having an I-shape and including a first beam, a second beam, and a vertical strut connecting the first beam with the second beam, the vertical strut defining a cam slot, the drive assembly movable between a drive retracted position and a drive advanced position; and
a tool assembly supported on the distal portion of the elongate body, the tool assembly defining a longitudinal axis and including an anvil and a cartridge assembly, the cartridge assembly including a channel defining a cavity and a staple cartridge that is releasably received within the cavity of the channel, the staple cartridge including:
a cartridge body defining a knife slot and staple receiving pockets, the staple receiving pockets positioned on opposite sides of the knife slot;
staples received within the staple receiving pockets of the cartridge body; and
an actuation sled assembly movable within the cartridge body from a sled retracted position to a sled advanced position to eject staples from the cartridge body of the staple cartridge, the actuation sled assembly including:
an actuation sled having a base portion, a central body portion extending upwardly from the base portion, and cam members positioned on opposite sides of the central body portion, the central body portion of the actuation sled defining a linear guide channel defining an axis that is transverse to the longitudinal axis of the tool assembly; and
a knife supported on the central body portion of the actuation sled and movable between a lowered position and a raised position, the knife including a first portion received within the linear guide channel of the actuation sled, a cutting blade, and a cam member, the cam member received within the cam slot of the vertical strut of the working member of the drive assembly when the drive assembly is moved from the drive retracted position towards the drive advanced position to move the knife from the lowered position to the raised position.

15. The surgical stapling device of claim 14, wherein the knife includes a detent, and the actuation sled includes a resilient arm that supports an abutment, the abutment is engaged with the detent when the knife is in the lowered position to retain the knife in the lowered position.

16. The surgical stapling device of claim 14, wherein the staple cartridge further includes a staple guard secured to a bottom of the cartridge body, the staple guard having a bottom wall engaged with the base portion of the actuation sled, and wherein the bottom wall of the staple guard includes a resilient tab, and the base portion of the actuation sled defines a recess, the resilient tab of the staple guard received within the recess of the actuation sled when the actuation sled assembly is in the sled advanced position to retain the actuation sled assembly in the sled advanced position.

17. The surgical stapling device of claim 14, wherein the base portion of the actuation sled defines a proximal cutout that is configured to receive the working member of the drive assembly when the drive assembly is moved from the drive retracted position towards the drive advanced position.

18. The surgical stapling device of claim 17, wherein the cam member of the knife is positioned over the cutout when the actuation sled assembly is in the sled retracted position.

19. The surgical stapling device of claim 14, wherein the vertical strut of the working member includes a distal surface that defines a recess, the cutting blade at least partially received within the recess during movement of the drive assembly from the drive retracted position towards the drive advanced position.

20. The surgical stapling device of claim 14, wherein the knife includes a central portion that defines a cavity, and the actuation sled includes spaced vertical walls that are received within the cavity of the central portion of the knife to confine the knife to linear movement as the knife moves between the raised and lowered positions.

21. A staple cartridge comprising:
a cartridge body defining a knife slot and staple receiving pockets, the staple receiving pockets positioned on opposite sides of the knife slot;
staples received within the staple receiving pockets of the cartridge body; and
an actuation sled assembly movable within the cartridge body from a sled retracted position to a sled advanced position to eject staples from the cartridge body of the staple cartridge, the actuation sled assembly including:
an actuation sled having a base portion, a central body portion extending upwardly from the base portion, and cam members positioned on opposite sides of the central body portion, the central body portion of the actuation sled defining a linear guide channel defining an axis that is transverse to the longitudinal axis of the tool assembly; and
a knife supported on the central body portion of the actuation sled and movable between a lowered position and a raised position, the knife including a first portion received within the linear guide channel of the actuation sled, a cutting blade, and a cam member, the cam member being positioned on a proximal portion of the knife.

22. The staple cartridge of claim 21, wherein the staple cartridge further includes a staple guard secured to a bottom of the cartridge body, the staple guard having a bottom wall engaged with the base portion of the actuation sled, and wherein the bottom wall of the staple guard includes a resilient tab, and the base portion of the actuation sled defines a recess, the resilient tab of the staple guard received within the recess of the actuation sled when the actuation sled assembly is in the sled advanced position to retain the actuation sled assembly in the sled advanced position.
